(19) United States
(12) Patent Application Publication
(10) Pub. No.: US 2013/0144168 A1
(43) Pub. Date: Jun. 6, 2013

(54) ULTRASOUND DIAGNOSTIC APPARATUS AND COMPUTER PROGRAM PRODUCT

(71) Applicant: Naoki YONEYAMA, Otawara-shi (JP)

(72) Inventor: Naoki YONEYAMA, Otawara-shi (JP)

(21) Appl. No.: 13/688,555

(22) Filed: Nov. 29, 2012

(30) Foreign Application Priority Data
Dec. 6, 2011 (JP) 2011-267034
Oct. 30, 2012 (JP) 2012-238621

(51) Int. Cl. A61B 8/00; A61B 8/08; A61B 8/14

(52) U.S. Cl. CPC A61B 8/4254; A61B 8/14; A61B 8/5292; A61B 8/4477
USPC 600/443; 600/437

(57) ABSTRACT

An ultrasound diagnostic apparatus according to an embodiment includes a memory unit and a converting unit. The memory unit stores therein conversion information, for each ultrasonic probe, converting a coordinate of an attachment position of a position sensor attached to an ultrasonic probe into a coordinate of a predefined position on a surface for transmitting and receiving ultrasonic waves on the ultrasonic probe. When the ultrasonic probe is replaced with another ultrasonic probe, the converting unit acquires conversion information corresponding to the replacing ultrasonic probe and uses the conversion information thus acquired to convert the coordinate of the attachment position of the position sensor attached to the replacing ultrasonic probe into the coordinate of the predefined position.

### CROSS-REFERENCE TO RELATED APPLICATIONS

This application is based upon and claims the benefit of priority from Japanese Patent Application No. 2011-267034, filed on Dec. 6, 2011; and Japanese Patent Application No. 2012-238621, filed on Oct. 30, 2012, the entire contents of all of which are incorporated herein by reference.

### FIELD

Embodiments described herein relate generally to an ultrasound diagnostic apparatus and a computer program product.

### BACKGROUND

Conventionally, an ultrasound diagnostic apparatus is used for periodic observations of patients having diseases associated with high risk of cancers as a non-invasive diagnostic apparatus. For example, the ultrasound diagnostic apparatus is used for periodic observations of patients having diseases associated with high risk of liver cancers such as hepatitis and cirrhosis.

In recent years, examinations using X-ray computed tomography (CT) apparatuses and magnetic resonance imaging (MRI) apparatuses have been conducted in conjunction with observations using the ultrasound diagnostic apparatus. The examinations using the X-ray CT apparatuses and the MRI apparatuses may detect lesions suspected of being cancerous in an examination using contrast agent, for example. There are an increasing number of cases where accurate diagnosis is established with the lesions thus detected undergoing fine needle cytology under ultrasound imaging.

An ultrasound diagnostic apparatus is known that includes an ultrasonic probe with a magnetic position sensor attached thereto and has a function of navigating the ultrasonic probe to the position of the lesion using the CT or MRI images with the lesions detected thereon as reference images. However, in the conventional technology, there are cases where diagnostic efficiency is decreased when the reference images are referred to in the diagnosis.

### BRIEF DESCRIPTION OF THE DRAWINGS

FIG. 1 is a diagram illustrating an overall structure of an ultrasound diagnostic apparatus according to a first embodiment;

FIG. 2 is a diagram illustrating an example of a structure of a position information acquiring device and a control unit according to the first embodiment;

FIG. 3 is a diagram illustrating a setting of a virtual sensor according to the first embodiment;

FIG. 4 is a flowchart illustrating procedures in processes performed by the ultrasound diagnostic apparatus according to the first embodiment;

FIG. 5 is a diagram illustrating a correspondence table between an ultrasonic probe and a position sensor according to a second embodiment; and

FIG. 6 is a diagram illustrating an overall structure of an ultrasound diagnostic apparatus according to the second embodiment.

### DETAILED DESCRIPTION

### First Embodiment

According to an embodiment, An ultrasound diagnostic apparatus comprising a memory unit and converting unit. The memory unit configured to store therein conversion information, for each ultrasonic probe, converting a coordinate of an attachment position of a position sensor attached to an ultrasonic probe into a coordinate of a predefined position on a surface for transmitting and receiving ultrasonic waves on the ultrasonic probe. The converting unit configured to, when the ultrasonic probe is replaced with another ultrasonic probe, acquire conversion information corresponding to the replacing ultrasonic probe from the memory unit and using the conversion information thus acquired to convert a coordinate of an attachment position of a position sensor attached to the replacing ultrasonic probe to the coordinate of the predefined position.

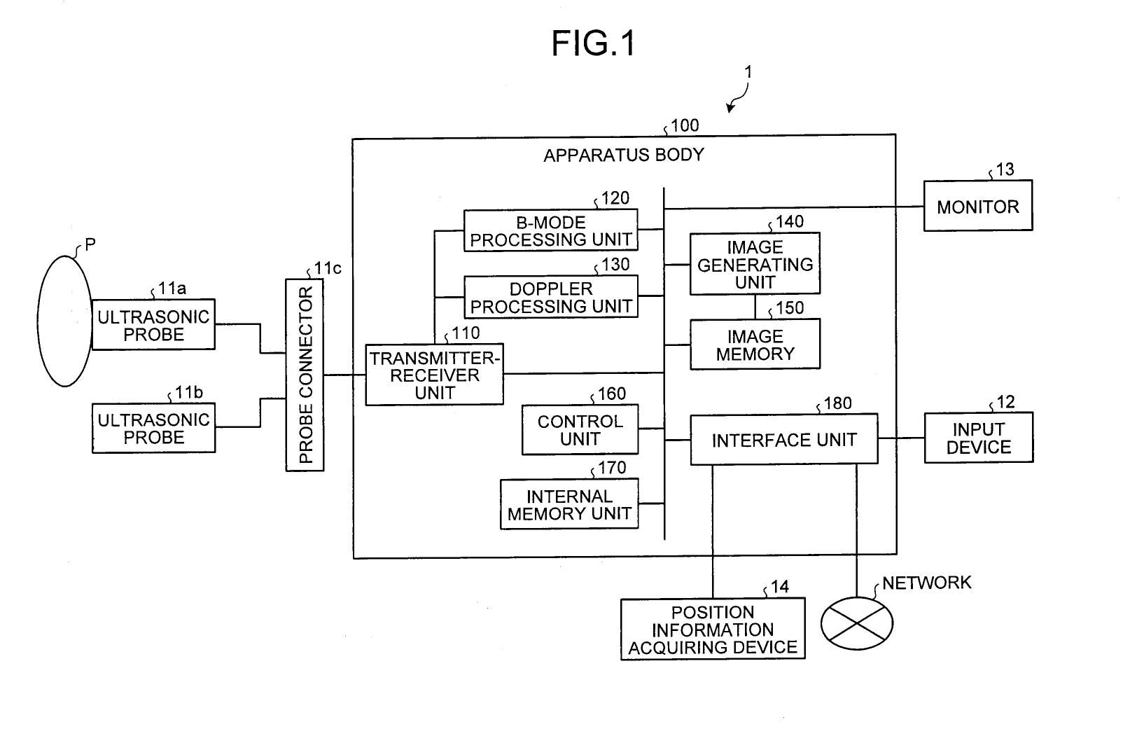

Firstly, an overall structure of an ultrasound diagnostic apparatus according to a first embodiment will be described with reference to FIG. 1. FIG. 1 is a diagram illustrating the overall structure of this ultrasound diagnostic apparatus 1 according to the first embodiment. As illustrated in FIG. 1, the ultrasound diagnostic apparatus 1 according to the first embodiment includes an ultrasonic probe 11a, an ultrasonic probe 11b, a probe connector 11c, an input device 12, a monitor 13, a position information acquiring device 14, and an apparatus body 100 and is connected with a network.

The ultrasonic probe 11a and the ultrasonic probe 11b each include a plurality of piezoelectric vibrators generating an ultrasonic wave based on a drive signal supplied from a transmitter-receiver unit 110 included in the apparatus body 100 described later. Also, the ultrasonic probe 11a and the ultrasonic probe 11b each receive a reflected wave from a subject P to convert it into an electrical signal. Furthermore, the ultrasonic probe 11a and the ultrasonic probe 11b each include a matching layer provided to the piezoelectric vibrators and a backing material preventing an ultrasonic wave from traveling behind the piezoelectric vibrators. For example, the types of the ultrasonic probe 11a and the ultrasonic probe 11b include a sector type, a linear type, and a convex type.

When ultrasonic waves are transmitted from the ultrasonic probe 11a or the ultrasonic probe 11b to the subject P, the ultrasonic waves transmitted are continuously reflected on a plane of discontinuity of acoustic impedances in body tissues of the subject P and then received by the plurality of piezoelectric vibrators included in the ultrasonic probe 11a or the ultrasonic probe 11b as reflected wave signals. The amplitude of the reflected wave signals received depends on the differences among the acoustic impedances on the plane of discontinuity on which the ultrasonic waves are reflected. It should be noted that when the ultrasonic pulses transmitted are reflected on the surface of a moving blood flow or cardiac wall, for example, the reflected wave signal undergoes a frequency shift depending on the velocity component against the ultrasound transmission direction of the moving body because of the Doppler effect.

The present embodiment is applicable to both the case where a subject P is two-dimensionally scanned with an ultrasonic probe 11a or an ultrasonic probe 11b that is a one-dimensional ultrasonic probe with a plurality of piezoelectric vibrators arranged in line, and the case where a subject P is three-dimensionally scanned with an ultrasonic probe 11a or an ultrasonic probe lib that mechanically oscillates the piezoelectric vibrators of the one-dimensional ultrasonic probe or with an ultrasonic probe 11a or an ultrasonic probe 11b that is a two-dimensional ultrasonic probe with a plurality of piezoelectric vibrators two-dimensionally arranged in a reticular pattern.

Furthermore, although FIG. 1 illustrates only two ultrasonic probes, embodiments are not limited to this arrangement. Any number of ultrasonic probes such as three or more ultrasonic probes may be included.

The probe connector 11c includes connectors to each of which the ultrasonic probe 11a and the ultrasonic probe 11b are connected and connects each of the ultrasonic probe 11a and the ultrasonic probe 11b to the apparatus body 100.

The input device 12 includes a trackball, a switch, buttons, and a touch command screen. The input device 12 receives various setting requests from an operator of the ultrasound diagnostic apparatus 1 and transmits the setting requests thus received to the apparatus body 100. For example, the input device 12 receives various operations related to positioning of ultrasonic images with other images such as X-ray CT images.

The monitor 13 displays a GUI (Graphical User Interface) for the operator of the ultrasound diagnostic apparatus 1 to input various setting requests using the input device 12 and displays in parallel ultrasonic images created in the apparatus body 100 with other images such as X-ray CT images, for example.

The position information acquiring device 14 acquires position information of the ultrasonic probe 11a and the ultrasonic probe lib. Specifically, the position information acquiring device 14 acquires position information that indicates where the ultrasonic probe 11a and the ultrasonic probe lib are positioned. The position information acquiring device 14 may be a magnetic sensor, an infrared sensor, an optical sensor, or a camera, for example.

The apparatus body 100 is an apparatus that generates an ultrasonic wave image based on a reflected wave received by the ultrasonic probe 11a or the ultrasonic probe 11b. As illustrated in FIG. 1, the apparatus body 100 includes the transmitter-receiver unit 110, a B-mode processing unit 120, a Doppler processing unit 130, an image generating unit 140, an image memory 150, a control unit 160, an internal memory unit 170, and an interface unit 180. The ultrasonic probe 11a and the ultrasonic probe 11b will be collectively referred to as an ultrasonic probe 11 in some cases below.

The transmitter-receiver unit 110 includes a trigger generation circuit, a delay circuit, and a pulsar circuit, and supplies drive signals to the ultrasonic probes 11. The pulsar circuit repeatedly generates a rate pulse for forming a transmission ultrasonic wave at a predefined rate frequency. Furthermore, the delay circuit converges ultrasonic waves generated from the ultrasonic probe 11 to a beam shape and provides each rate pulse generated by the pulsar circuit with a delay time for each piezoelectric vibrator required to determine transmission directionality. The trigger generation circuit applies a drive signal (drive pulse) to the ultrasonic probe 11 at the timing based on the rate pulse. In other words, the delay circuit adjusts the transmission direction from the surface of the piezoelectric vibrators as required by changing the delay time provided to each rate pulse.

The transmitter-receiver unit 110 includes an amplifier circuit, an A/D (analog/digital) converter, and an adder, and generates reflected wave data through various processes on a reflected wave signal received by the ultrasonic probe 11. The amplifier circuit amplifies the reflected wave signal for each channel to perform a gain correction process. The A/D converter A/D-converts the reflected wave signal thus gain-corrected and provides a delay time required to determine reception directionality. The adder performs an adding process of the reflected wave signal processed by the A/D converter to generate reflected wave data. The adding process performed by the adder emphasizes reflection components from the direction in accordance with the reception directionality of the reflected wave signal.

Thus, the transmitter-receiver unit 110 controls transmission and reception directionalities in the transmission and reception of the ultrasonic wave. It should be noted that the transmitter-receiver unit 110 has a function of instantaneously changing delay information, transmission frequencies, transmission drive voltages, the numbers of aperture elements, for example, under the control of the control unit 160 described later. In particular, changing transmission drive voltages is achieved by a linear amplifier type of oscillation circuit capable of instantaneously changing values or a mechanism electrically changing over a plurality of power source units. In addition, the transmitter-receiver unit 110 is capable of transmitting and receiving different waveforms for each frame or each rate.

The B-mode processing unit 120 receives reflected wave data being reflected wave signals after processes of gain correction, A/D conversion, and adding from the transmitter-receiver unit 110 and performs logarithmic amplification, envelope demodulation, and the like to generate data in which the intensity of a signal is represented by the brightness of its luminance (B-mode data).

The Doppler processing unit 130 performs frequency analysis of velocity information from the reflected wave data received from the transmitter-receiver unit 110 and extracts blood flows, tissues, and contrast agent echo components influenced by the Doppler effect, generating data formed of extraction of moving body information in many aspects such as average velocity, variance, and power.

The image generating unit 140 generates an ultrasonic image from the B-mode data generated by the B-mode processing unit 120 and the Doppler data generated by the Doppler processing unit 130. Specifically, the image generating unit 140 converts (scan-converts) a row of scan line signals from an ultrasonic scanning into a row of scan line signals in a video format represented by television, for example, to generate an ultrasonic wave image (B-mode image or Doppler image) as an image for display from B-mode data or Doppler data. Furthermore, the image generating unit 140 generates a two-dimensional image from volume data in different modalities stored in the internal memory unit 170 under the control of the control unit described later.

The image memory 150 stores therein image data such as angiographic and histological pictures generated by the image generating unit 140. The image memory 150 also stores therein results of the processes performed by the image generating unit 140 described later. Furthermore, the image memory 150 stores output signals (RF: radio frequencies) having just passed through the transmitter-receiver unit 110, luminance signals of images, various raw data, image data acquired via a network, for example, as necessary. The data format of the image data stored in the image memory 150 may be either the data format of the video format displayed on the monitor 13 after conversion under the control of the control unit 160 described later or the data format of the raw data before coordinate conversion generated by the B-mode processing unit 120 and the Doppler processing unit 130.

The control unit 160 controls the overall processes performed by the ultrasound diagnostic apparatus 1. Specifically, the control unit 160 controls processes performed by the transmitter-receiver unit 110, the B-mode processing unit 120, the Doppler processing unit 130, and the image generating unit 140 based on various setting requests input by an operator via the input device 12 and various control programs and setting information read out from the internal memory unit 170. The control unit 160 also controls to display an ultrasonic wave image stored in the image memory 150, for example, on the monitor 13.

The internal memory unit 170 stores therein various types of data such as a control program for performing transmission and reception of an ultrasonic wave, image processing, and display processing, diagnostic information (patients' IDs and doctors' opinions, for example), a diagnostic protocol, and the like. The internal memory unit 170 is also used for storing therein images stored in the image memory 150 as necessary. Furthermore, the internal memory unit 170 stores therein various types of information used for the processes performed by the control unit 160. The information will be described later.

The interface unit 180 is an interface controlling communication of various types of information among the input device 12, the position information acquiring device 14, the network, and the apparatus body 100. For example, the interface unit 180 controls forwarding of position information acquired by the position information acquiring device 14 to the control unit 160.

The overall structure of the ultrasound diagnostic apparatus according to the first embodiment has been described above. Based on such a structure, the ultrasound diagnostic apparatus 1 according to the first embodiment is configured to be able to improve diagnostic efficiency in diagnosis performed with the processes of the position information acquiring device 14 and the control unit 160 described later with reference to reference images.

Positioning of images for diagnosis performed using CT or MRI images as reference images will now be described. When diagnosis is performed using CT or MRI images as reference images, volume data in an X-ray CT apparatus or an MRI apparatus can be associated with an ultrasonic image using a magnetic sensor attached to the ultrasonic probe, for example.

Firstly, three axes (X, Y, Z) in the magnetic field of the ultrasonic probe with the magnetic sensor attached thereto are aligned with three axes of volume data in other modality. Specifically, vertically put the ultrasonic probe with the magnetic sensor attached thereto on the subject and in that state, press down the set button to set the orientation of the magnetic sensor at that time to vertical.

Next, select an ultrasonic image presenting the same characteristic part as that presented in the image in the other modality and press the set button again, associating the position (coordinate) of the magnetic sensor at that time with the position (coordinate) in the volume data in the other modality. For the characteristic part, a blood vessel or an ensiform cartilage, for example, is used.

As described above, associating the orientation and the coordinate of the magnetic sensor with the coordinate of the volume data in the other modality enables generating a two-dimensional image of the position substantially same as the face currently scanned by the ultrasonic probe from the volume data in the other modality. Furthermore, when registering a lesion suspected of being cancerous detected in the image in the other modality as a range of tumor, a mark is placed on the substantially same position on the ultrasonic image. A physician performs needling based on the mark.

However, in many cases, the ultrasonic probe for identifying the position of a lesion is different from the probe for performing needling. For example, for the ultrasonic probe for identifying the position of a lesion, an ultrasonic probe having a wide surface for transmitting and receiving ultrasonic waves is used in order to acquire accurate images. In contrast, for the probe for performing needling, an ultrasonic probe having a narrow surface for transmitting and receiving ultrasonic waves is used in order to find a narrow gap easily.

For example, the magnetic sensor can be attached to the ultrasonic probe by attaching the magnetic sensor to a magnetic sensor holder provided on the surface of the ultrasonic probe. When the magnetic sensor holder is attached on the boundary between the ultrasonic probe and a cable, for example, the position of the magnetic sensor attached is to be on the root of the cable for the ultrasonic probe. However, because the shapes of ultrasonic probes depend on each item, the position for attaching the magnetic sensor is not always the same. Accordingly, in a case where positioning is performed by associating the coordinate of the magnetic sensor with the coordinate of the volume data in the other modality, changing the ultrasonic probe will cause a position gap.

Therefore, in the conventional technology, every time an ultrasonic probe is changed, positioning described above has to be performed, decreasing diagnostic efficiency in diagnosis performed with reference to reference images. Thus, the ultrasound diagnostic apparatus 1 according to the first embodiment is configured to be able to improve diagnostic efficiency in diagnosis performed with reference to reference images by eliminating the need of positioning in accordance with changing ultrasonic probes.

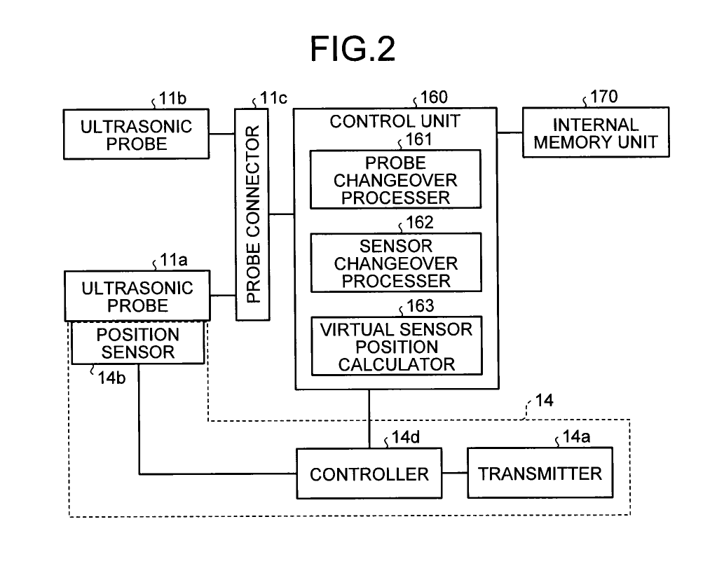

The processes performed by the position information acquiring device 14 and the control unit 160 according to the first embodiment will be described below with reference to FIG. 2, for example. FIG. 2 is a diagram illustrating an example of the structure of the position information acquiring device 14 and the control unit 160 according to the first embodiment. The position information acquiring device 14 according to the first embodiment includes a transmitter 14a, a position sensor 14b, and a controller 14d and is connected to the control unit 160 via the interface unit 180, which is not illustrated, as illustrated in FIG. 2.

The transmitter 14a is placed on an optional position and forms a magnetic field extended to the outside centering on itself. The position sensor 14b is mounted on the surface of the ultrasonic probe 11a and detects the three-dimensional magnetic field formed by the transmitter 14a. The position sensor 14b converts information of the magnetic field thus detected into signals and outputs the signals to the controller 14d.

The controller 14d calculates the coordinate and the orientation of the position sensor 14b in a space with its origin at the transmitter 14a based on the signals received from the position sensor 14b, and outputs the coordinate and the orientation thus calculated to the control unit 160. Diagnosis of the patient P is performed within a magnetic field area where the position sensor 14b mounted on the ultrasonic probe 11a can accurately detect the magnetic field of the transmitter 14a.

The control unit 160 includes a probe changeover processor 161, a sensor changeover processor 162, and a virtual sensor position calculator 163 and is connected to the position information acquiring device 14 and the internal memory unit 170 via a bus and the interface unit 180, which are not illustrated.

The internal memory unit 170 stores therein various types of information used by the probe changeover processor 161, the sensor changeover processor 162, and the virtual sensor position calculator 163. Specifically, the internal memory unit 170 stores therein information on the position sensor 14b and the ultrasonic probe 11b and information on a virtual sensor. For example, the internal memory unit 170 stores therein ultrasonic probe names, probe IDs, information on the position to which the position sensor is attached, and information on visual field depth.

Furthermore, the internal memory unit 170 stores therein the virtual sensor information used for calculating the virtual sensor position that is a coordinate associating the ultrasonic image and the volume data in the other modality. For example, the internal memory unit 170 stores therein virtual sensor information such as (ΔSx1, 0, −ΔSz1) and (ΔSx2, 0, −ΔSz2) for each ultrasonic probe. Description of each coordinate will be made later.

The probe changeover processor 161 acquires information on ultrasonic probes from the internal memory unit 170 upon receiving an ultrasonic probe changeover process from the operator through the input device 12. For example, when the probe changeover processor 161 receives a changeover process from the ultrasonic probe 11a to the ultrasonic probe 11b, the probe changeover processor 161 acquires the ultrasonic wave probe name and probe ID of the ultrasonic probe 11b, information on the position to which the position sensor is attached, and information on visual field depth. Thereafter, the probe changeover processor 161 controls the ultrasonic probe 11b. Furthermore, the probe changeover processor 161 outputs the information thus acquired to the sensor changeover processor 162 or the virtual sensor position calculator 163.

The sensor changeover processor 162 acquires information on the position sensor from the internal memory unit 170 upon receiving information of the probe change from the probe changeover processor 161. For example, when the sensor changeover processor 162 receives information of the change from the ultrasonic probe 11a to the ultrasonic probe 11b, the sensor changeover processor 162 acquires information on the position sensor 14b. Thereafter, the sensor changeover processor 162 controls the position sensor 14b.

The virtual sensor position calculator 163 acquires information on the virtual sensor from the internal memory unit 170 upon receiving information of the ultrasonic probe change. Thereafter, the virtual sensor position calculator 163 calculates virtual sensor offset position information for offsetting the virtual sensor using the visual field depth and the virtual sensor information received from the probe changeover processor 161. The virtual sensor position calculator 163 is also referred to as a converting unit.

The virtual sensor will now be described. The virtual sensor is three-dimensional coordinate data set by the controller 14d and is used for indicating the center positions of sections of reference images such as X-ray CT or MRI images. If the intersection with which a vector perpendicularly intersects with a plane is known at this point, the relationship among the plane, the vector, and the inner product is used for defining the plane. Because a coordinate system defined by the position sensor is different from a coordinate system defined by a reference image, the determinant representing the relationship between the two can be obtained with a known formula.

In the ultrasound diagnostic apparatus 1 according to the first embodiment, the coordinate of the virtual sensor is associated with the coordinate of the volume data. In the present embodiment, the virtual sensor is set so that an ultrasonic probe can be used without positioning even if change of ultrasonic probes is performed. In other words, the virtual sensor is set with consideration for the position to which the position sensor is attached, which depends on each ultrasonic probe.

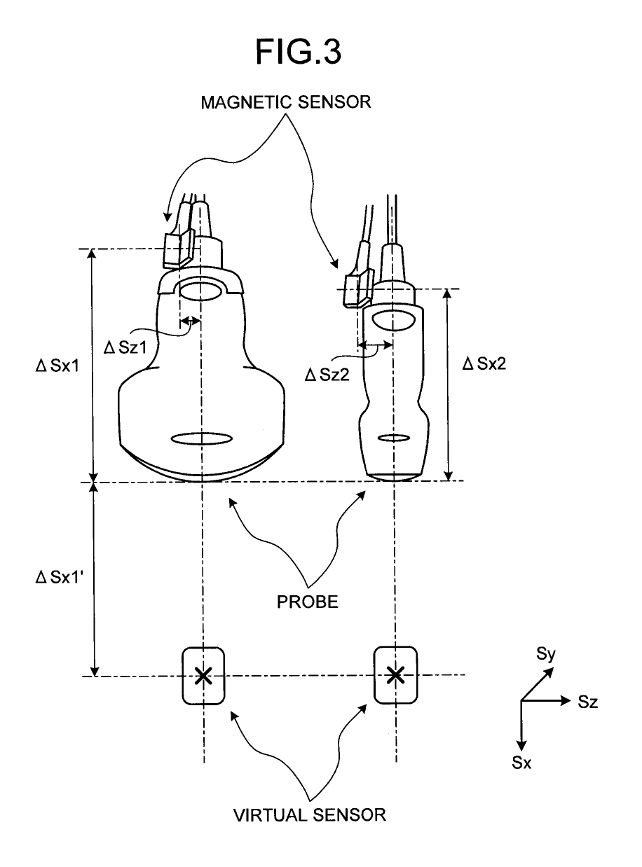

Setting of the virtual sensor will be described below with reference to FIG. 3. FIG. 3 is a diagram illustrating setting of the virtual sensor according to the first embodiment. In FIG. 3, different ultrasonic probes are placed in a three-dimensional space. For example, in the setting of the virtual sensor, the coordinate of the position sensor is converted into the coordinate of the center of the surface for transmitting and receiving ultrasonic waves in an ultrasonic probe. In other words, the coordinate of the center of the surface for transmitting and receiving ultrasonic waves in an ultrasonic probe can be set as the virtual sensor. In such a case, in the ultrasonic probe illustrated on the left in FIG. 3 for example, the distance from the position of the position sensor to the coordinate of the center of the surface for transmitting and receiving ultrasonic waves (the coordinate of the virtual sensor) is to be “ΔSx1” in the positive direction on the X axis and “0” on the Y axis, and “ΔSz1” in the negative direction on the Z axis. More specifically, the virtual sensor can be obtained through conversion of (x, y, z)=(ΔSx1, 0, −ΔSz1) performed with relation to the coordinate detected by the position sensor. It should be noted that the coordinate of the position sensor attachment position with relation to the coordinate of the center of the surface for transmitting and receiving ultrasonic waves can be obtained from the product specification, for example.

Similarly, in the ultrasonic probe illustrated on the right in FIG. 3, the virtual sensor can be obtained through conversion of (x, y, z)=(ΔSx2, 0, −ΔSz2) performed with relation to the coordinate detected by the position sensor. It should be noted that the position sensor is attached so that the angle detected by the position sensor is used.

Furthermore, information on the visual field depth can be reflected with consideration for easiness of image display on the screen. More specifically, in order to control the center of the image to be displayed on the center of the screen, the coordinate of the virtual sensor described above is further converted. In the ultrasound diagnostic apparatus, the center of the displayed image on the screen is to be the position at ½ of the visual field depth. Accordingly, the value of ½ of the visual field depth value is added.

In the present embodiment, the virtual sensor is set on the position corresponding to the position of ½ of the ultrasonic probe caliber and the position of ½ of the visual field depth so that the virtual sensor is positioned on the center of the image on the screen. However, the position of the virtual sensor is not limited to this example, but may be set to the position of ½ of the ultrasonic probe caliber on the surface for transmitting and receiving ultrasonic waves (that is the position where the visual field depth is 0). Alternatively, the position of the virtual sensor may be set with the input device 12 so that the virtual sensor is positioned on an optional position on the screen.

For example, in the case where “ΔSx1′”=“½×L, (visual field depth)” as illustrated in FIG. 3, the virtual sensor described above (x, y, z)=(ΔSx1, 0, −ΔSz1) will be (x, y, z)=(ΔSx1+ΔSx1′, 0, −ΔSz1). The value of the virtual sensor described above is calculated by the virtual sensor position calculator 163 and output to the controller 14d as virtual sensor offset information. The controller 14d sets the virtual sensor using the virtual sensor offset information received.

Furthermore, when changing to the ultrasonic probe illustrated on the right in FIG. 3 for example, the virtual sensor position calculator 163 reads out the virtual sensor information (x, y, z)=(ΔSx2, 0, −ΔSz2) from the internal memory unit 170. Thereafter, the virtual sensor position calculator 163 calculates (x, y, z)=(ΔSx2+ΔSx1′, 0, −ΔSz2) that is the virtual sensor information read out (x, y, z)=(ΔSx2, 0, −ΔSz2) added with “ASx1′” and output the information thus calculated to the controller 14d.

The processes described above enables the position of the virtual sensor to remain unchanged even if the ultrasonic probe is changed. Thus, no position gap is caused between the ultrasonic image and the volume data in the other modality, eliminating the need for positioning in diagnosis performed with reference to reference images.

Specifically, the control unit 160 controls the image generating unit 140 to generate a two-dimensional image from the volume data in the other modality based on the change of the coordinate of the virtual sensor.

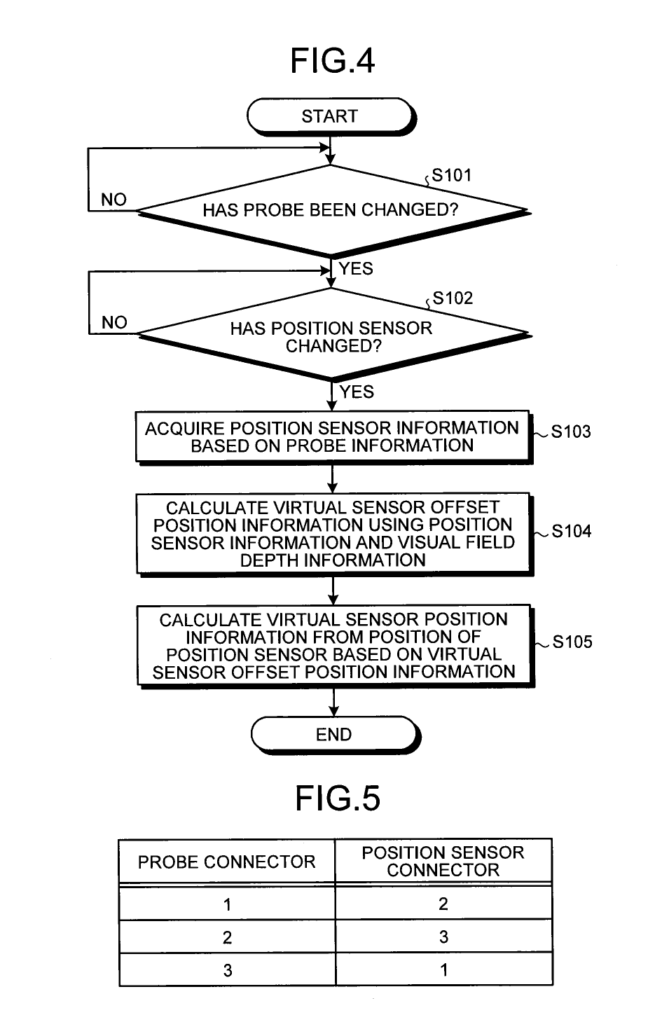

Next, processes performed by the ultrasound diagnostic apparatus 1 according to the first embodiment will be described. FIG. 4 is a flowchart illustrating procedures in the processes performed by the ultrasound diagnostic apparatus 1 according to the first embodiment. It should be noted that FIG. 4 illustrates processes in the case where diagnosis of the subject P is performed in a magnetic field area in which the position sensor 14b can accurately detect the magnetic field of the transmitter 14a.

As illustrated in FIG. 4, in the ultrasound diagnostic apparatus 1 according to the first embodiment, when the ultrasonic probe is changed (Yes at Step S101), the sensor changeover processor 162 determines if the position sensor is changed (Step S102).

If the position sensor is changed (Yes at Step S102), the sensor changeover processor 162 acquires the position sensor information based on ultrasonic probe information (Step S103). In contrast, if the position sensor is not changed (No at Step S102), the sensor changeover processor 162 is in a wait state.

Furthermore, the virtual sensor position calculator 163 calculates the virtual sensor offset position information using the position sensor information and the visual field depth information (Step S104). Thereafter, the controller 14d calculates the position information of the virtual sensor from the position of the position sensor based on the virtual sensor offset position information received from the virtual sensor position calculator 163 (Step S105), completing the processes.

As described above, in the first embodiment, the internal memory unit 170 stores therein information of the conversion for each ultrasonic probe in which the coordinate of the attachment position of the position sensor attached to the ultrasonic probe is converted into the coordinate of the predefined position on the surface for transmitting and receiving ultrasonic waves on the ultrasonic probe. The virtual sensor position calculator 163 acquires conversion information corresponding to the replacing ultrasonic probe from the internal memory unit 170 when change of the ultrasonic probes is performed, and converts the coordinate of the attachment position of the position sensor attached to the replacing ultrasonic probe into the coordinate of the predefined position using the conversion information thus acquired. Thus, the ultrasound diagnostic apparatus 1 according to the first embodiment can perform diagnosis with reference to reference images without positioning in accordance with change of ultrasonic probes and improve diagnosis efficiency in diagnosis performed with reference to reference images.

In the first embodiment, the virtual sensor position calculator 163 further converts the coordinate after the conversion above in the position of half the ultrasonic probe caliber based on the value of ½ of the visual field depth. Thereafter, the control unit 160 controls to generate a two-dimensional image from the three-dimensional image in accordance with the change of the coordinate after the conversion based on the value of ½ of the visual field depth. Thus, the ultrasound diagnostic apparatus 1 according to the first embodiment can control the center of the image to be positioned on the center of the screen and improve the visibility of the image.

Furthermore, in the first embodiment, the position sensor 14b is shared for a plurality of ultrasonic probes. Accordingly, the ultrasound diagnostic apparatus 1 according to the first embodiment can flexibly change the ultrasonic probes.

### Second Embodiment

The first embodiment has been described above. However, besides the first embodiment described above, various embodiments may be performed.

### (1) Sensor Changeover Process

In the first embodiment described above, a case where one position sensor is shared for a plurality of ultrasonic probes is described. However, embodiments are not limited to the structure described above but may be applicable to the structure in which each of the ultrasonic probes is provided with a position sensor, for example. In such a case, when an ultrasonic probe is changed, the position sensor is changed as well. Position sensors may be changed both manually and automatically.

FIG. 5 is a diagram illustrating a correspondence table between an ultrasonic probe and a position sensor according to a second embodiment. For example, when the change of the position sensor is performed automatically, the internal memory unit 170 stores therein the correspondence table indicating the correspondence between the ultrasonic probe and the position sensor attached thereto. For example, as illustrated in FIG. 5, the internal memory unit 170 stores therein the correspondence table indicating the correspondence between the ultrasonic probe and the position sensor attached thereto with the numbers of connectors therefor. When the sensor changeover processor 162 receives information of ultrasonic probe change, the sensor changeover processor 162 refers to the correspondence table to perform the process of changing the position sensor.

### (2) Volume Data

In the first embodiment described above, a case where volume data generated by an X-ray CT apparatus or an MRI apparatus is used is described. However, embodiments are not limited to the structure described above but may be applicable to the structure in which volume data generated by the ultrasound diagnostic apparatus is used.

### (3) Virtual Sensor Information

In the first embodiment above, a case where the virtual sensor information for each ultrasonic probe is stored in the internal memory unit 170 provided in the ultrasound diagnostic apparatus 1 has been described. However, embodiments are not limited to the structure above but applicable to a case where the virtual sensor information is stored in an external memory device.

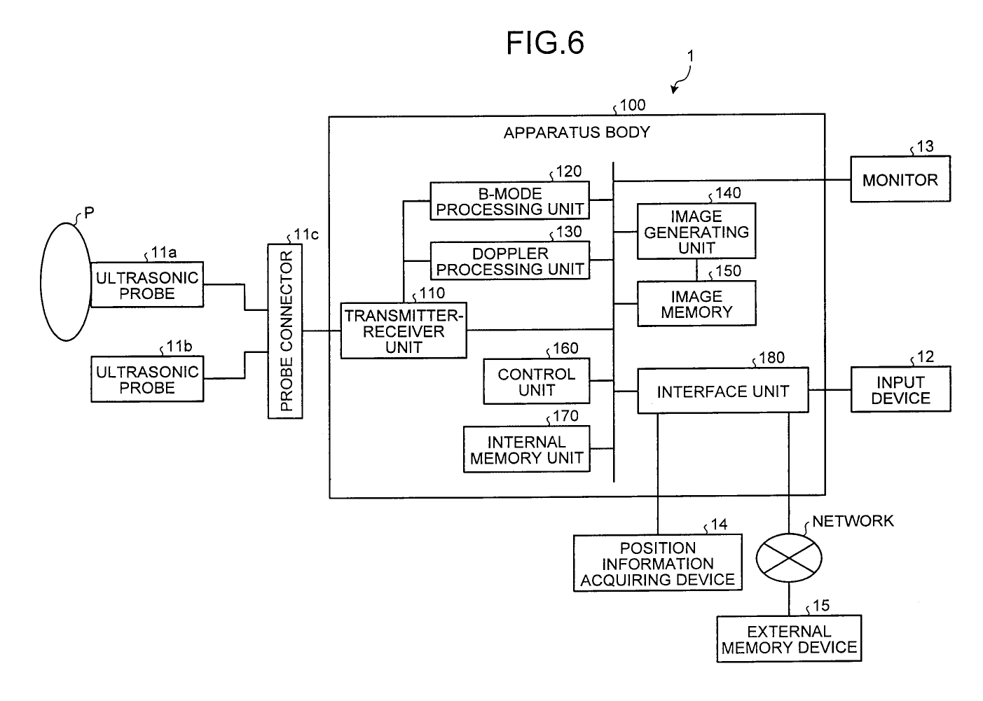

FIG. 6 is a diagram illustrating the overall structure of an ultrasound diagnostic apparatus 1 according to the second embodiment. As illustrated in FIG. 6, the ultrasound diagnostic apparatus 1 according to the second embodiment is connected to an external memory device 15 via a network, to which a picture archiving and communication system (PACS), hospital information system (HIS), and radiology information system (RIS) are applicable, for example.

The external memory device 15 stores therein information on conversion to the coordinate of a predefined position on the surface for transmitting and receiving ultrasonic waves on the ultrasonic probe for each ultrasonic probe. For example, the external memory device 15 stores therein virtual sensor information such as (ΔSx1, 0, −ΔSz1) and (ΔSx2, 0, −ΔSz2).

The interface unit 180 is connected to the external memory device 15 storing therein the virtual sensor via the network. The interface unit 180 is also referred to as a connecting unit. When an ultrasonic probe is replaced with another ultrasonic probe, the virtual sensor position calculator 163 acquires conversion information corresponding to the replacing ultrasonic probe from the memory unit through the connecting unit, and uses the conversion information thus acquired to convert the coordinate of the attachment position of the position sensor attached to the replacing ultrasonic probe to the coordinate of the predefined position.

Specifically, the virtual sensor position calculator 163 acquires virtual sensor information corresponding to the replacing ultrasonic probe from the external memory device 15 via the interface unit 180 when the ultrasonic probe is changed and calculates virtual sensor offset position information.

Thereafter, the virtual sensor position calculator 163 outputs the virtual sensor offset position information thus calculated to the controller 14d. The controller 14d sets the virtual sensor based on the virtual sensor offset position information thus received.

The external memory device 15 can store therein not only the virtual sensor information but information on the ultrasonic probes and the position sensor such as the ultrasonic probe names, prove IDs, information of the virtual sensor attachment position, and the visual field depth. In such a case, the probe changeover processor 161 and the sensor changeover processor 162 acquire various types of information through the interface unit 180.

### (4) Angle Of The Position Sensor

In the first embodiment above, a case where the virtual sensor information based on the coordinate of the position sensor attachment position is used has been described. However, embodiments are not limited to the structure above but applicable to a case where virtual sensor information further considering the position sensor attachment angle is used. In such a case, the internal memory unit 170 stores therein virtual sensor information of each attachment angle of the position sensor (magnetic sensor, for example) attached to the ultrasonic probe for each ultrasonic probe, for example.

When the ultrasonic probe is replaced with another ultrasonic probe, the virtual sensor position calculator 163 acquires virtual sensor information corresponding to the replacing ultrasonic probe and the position sensor attachment angle from the internal memory unit 170, and uses the virtual sensor information thus acquired to convert the coordinate of the attachment position of the position sensor (magnetic sensor, for example) attached to the replacing ultrasonic probe to the coordinate of the predefined position. More specifically, the virtual sensor position calculator 163 uses the virtual sensor information thus acquired to calculate the virtual sensor offset position information and outputs the virtual sensor offset position information thus calculated to the controller 14d.

The virtual sensor information for each position sensor attachment angle described above is not only stored in the internal memory unit 170 but may be stored in the external memory device 15.

With an ultrasound diagnostic apparatus according to at least one of the embodiments described above, diagnostic efficiency can be improved in diagnosis performed with reference to reference images.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic apparatus comprising:
a memory unit configured to store therein conversion information, for each ultrasonic probe, converting a coordinate of an attachment position of a position sensor attached to an ultrasonic probe into a coordinate of a predefined position on a surface for transmitting and receiving ultrasonic waves on the ultrasonic probe; and
a converting unit configured to, when the ultrasonic probe is replaced with another ultrasonic probe, acquire conversion information corresponding to the replacing ultrasonic probe from the memory unit and using the conversion information thus acquired to convert a coordinate of an attachment position of a position sensor attached to the replacing ultrasonic probe to the coordinate of the predefined position.

2. The ultrasound diagnostic apparatus according to claim 1, further comprising:
setting unit configured to set the coordinate of the predefined position to a predefined reference position with relation to the surface for transmitting and receiving ultrasonic waves; and
controlling unit configured to control to generate a two-dimensional image from three-dimensional image data, wherein
the converting unit further converts the coordinate of the predefined position after the conversion based on the coordinate of the reference position, and
the controlling unit controls to generate a two-dimensional image from three-dimensional image data based on the coordinate of the reference position.

3. The ultrasound diagnostic apparatus according to claim 2, wherein the setting unit sets a position of half the caliber of the ultrasonic probe that is a position of ½ of visual field depth as the reference position.

4. The ultrasound diagnostic apparatus according to claim 2, wherein
the setting unit sets a position of half the caliber of the ultrasonic probe that is a position on a scan surface of the ultrasonic probe as the reference position.

5. The ultrasound diagnostic apparatus according to claim 2, further comprising:
inputting unit configured to input an optional position with relation to the surface for transmitting and receiving ultrasonic waves, wherein
the setting unit sets a position input by the inputting unit as the reference position.

6. The ultrasound diagnostic apparatus according to claim 1, wherein the position sensor is shared for a plurality of ultrasonic probes.

7. The ultrasound diagnostic apparatus according to claim 1, wherein a position sensor is attached to each of a plurality of ultrasonic probes and the position sensors are provided so as to be manually changeable in accordance with change of the ultrasonic probes.

8. The ultrasound diagnostic apparatus according to claim 1, further comprising:
changing unit configured to perform change of the position sensor, wherein
the memory unit stores therein information of position sensors corresponding to the respective ultrasonic probes, and
the changing unit performs change of the position sensor in accordance with change of the ultrasonic probes based on the information of position sensors corresponding to the respective ultrasonic probes stored in the memory unit.

9. The ultrasound diagnostic apparatus according to claim 1, wherein
the memory unit stores therein the conversion information for each of the ultrasonic probes and for each attachment angle of position sensors attached to the ultrasonic probes, and
the converting unit, when the ultrasonic probe is replaced with another ultrasonic probe, acquires conversion information corresponding to the replacing ultrasonic probe and an attachment angle of the position sensor corresponding thereto from the memory unit and uses the conversion information thus acquired to convert the coordinate of the attachment position of the position sensor attached to the replacing ultrasonic probe to the coordinate of the predefined position.

10. An ultrasound diagnostic apparatus comprising:
a connecting unit configured to connect to a memory unit storing therein conversion information, for each ultrasonic probe, converting a coordinate of an attachment position of a position sensor attached to an ultrasonic probe into a coordinate of a predefined position on a surface for transmitting and receiving ultrasonic waves on the ultrasonic probe; and
a converting unit configured to, when the ultrasonic probe is replaced with another ultrasonic probe, acquire conversion information corresponding to the replacing ultrasonic probe from the memory unit through the connecting unit and using the conversion information thus acquired to convert a coordinate of an attachment position of a position sensor attached to the replacing ultrasonic probe to the coordinate of the predefined position.

11. The ultrasound diagnostic apparatus according to claim 10, further comprising:
setting unit configured to set the coordinate of the predefined position to a predefined reference position with relation to the surface for transmitting and receiving ultrasonic waves; and
controlling unit configured to control to generate a two-dimensional image from three-dimensional image data, wherein
the converting unit further converts the coordinate of the predefined position after the conversion based on the coordinate of the reference position, and
the controlling unit controls to generate a two-dimensional image from three-dimensional image data based on the coordinate of the reference position.

12. The ultrasound diagnostic apparatus according to claim 11, wherein the setting unit sets a position of half the caliber of the ultrasonic probe that is a position of ½ of visual field depth as the reference position.

13. The ultrasound diagnostic apparatus according to claim 11, wherein
the setting unit sets a position of half the caliber of the ultrasonic probe that is a position on a scan surface of the ultrasonic probe as the reference position.

14. The ultrasound diagnostic apparatus according to claim 11, further comprising:
inputting unit configured to input an optional position with relation to the surface for transmitting and receiving ultrasonic waves, wherein
the setting unit sets a position input by the inputting unit as the reference position.

15. The ultrasound diagnostic apparatus according to claim 10, wherein the position sensor is shared for a plurality of ultrasonic probes.

16. The ultrasound diagnostic apparatus according to claim 10, wherein a position sensor is attached to each of a plurality of ultrasonic probes and the position sensors are provided so as to be manually changeable in accordance with change of the ultrasonic probes.

17. The ultrasound diagnostic apparatus according to claim 10, further comprising:
changing unit configured to perform change of the position sensor, wherein
the memory unit stores therein information of position sensors corresponding to the respective ultrasonic probes, and
the changing unit performs change of the position sensor in accordance with change of the ultrasonic probes based on the information of position sensors corresponding to the respective ultrasonic probes stored in the memory unit.

18. The ultrasound diagnostic apparatus according to claim 10, wherein
the connecting unit is connected to the memory unit storing therein the conversion information for each of the ultrasonic probes and for each attachment angle of position sensors attached to the ultrasonic probes, and
the converting unit, when the ultrasonic probe is replaced with another ultrasonic probe, acquires conversion information corresponding to the replacing ultrasonic probe and an attachment angle of the position sensor corresponding thereto from the memory unit through the connecting unit and uses the conversion information thus acquired to convert the coordinate of the attachment position of the position sensor attached to the replacing ultrasonic probe to the coordinate of the predefined position.

19. A computer program product capable of running on a computer and having a non-transitory computer readable recording medium including a plurality of commands for converting coordinates, wherein the commands cause the computer to perform:
acquiring, when an ultrasonic probe is replaced with another ultrasonic probe, conversion information corresponding to the replacing ultrasonic probe from a memory unit storing therein conversion information, for each ultrasonic probe, converting a coordinate of an attachment position of a position sensor attached to the ultrasonic probe into a coordinate of a predefined position on a surface for transmitting and receiving ultrasonic waves on the ultrasonic probe; and
using the conversion information thus acquired to convert the coordinate of the attachment position of the position sensor attached to the replacing ultrasonic probe into the coordinate of the predefined position.
